(19) United States
(12) Patent Application Publication
(10) Pub. No.: US 2026/0069783 A1
(43) Pub. Date: Mar. 12, 2026

(54) Medicament Container Carrier and Adapter

(71) Applicant: SANOFI-AVENTIS DEUTSCHLAND GMBH, Frankfurt am Main (DE)

(72) Inventors: Derek Fenlon, Wexford (IE); Pascal Launois, Dublin (IE); Julian McDonnell, Co. Wicklow (IE); Martina Moyne, Co. Donegal (IE); Conor Mulcahy, Co. Dublin (IE)

(21) Appl. No.: 19/388,368

(22) Filed: Nov. 13, 2025

Related U.S. Application Data
(63) Continuation of application No. 18/957,952, filed on Nov. 25, 2024, now Pat. No. 12,491,317.
Application No. 18/957,952 is a continuation of application No. 17/564,354, filed on Dec. 29, 2021, now Pat. No. 12,420,015.
Application No. 17/564,354 is a continuation of application No. 14/775,316, now Pat. No. 11,235,102.

(30) Foreign Application Priority Data
Mar. 14, 2013 (EP) 13159253.7

(51) Int. Cl. A61M 5/19; A61J 1/20; A61M 5/31

(52) U.S. Cl. CPC A61M 5/19; A61J 1/2096; A61M 5/3129; A61J 1/201

(57) ABSTRACT

Described is a medicament container carrier adapted to hold a first medicament container having a first predetermined size and capable of containing more than a first volume of a medicament. The container carrier comprises a body, and an adapter coupled to the body. The adapter is adapted to hold a second medicament container having a second predetermined size different from the first predetermined size and capable of containing no more than the first volume of the medicament.

### CROSS-REFERENCE TO RELATED APPLICATIONS

This application is a continuation application of U.S. application Ser. No. 18/957,952, filed on Nov. 25, 2024, which is a continuation application of U.S. application No. Ser. No. 17/564,354, filed on Dec. 29, 2021, now U.S. Pat. No. 12,420,015, which is a continuation application of U.S. application Ser. No. 14/775,316, filed on Sep. 11, 2015, now U.S. Pat. No. 11,235,102, which is a U.S. national stage application under 35 USC § 371 of International Application No. PCT/EP2014/054922, filed on Mar. 13, 2014, which claims priority to European Patent Application No. 13159253.7, filed on Mar. 14, 2013, the entire contents of which are incorporated herein by reference.

### TECHNICAL FIELD

The invention relates to an adapter for a medicament container carrier.

### BACKGROUND OF THE INVENTION

In a conventional medicament delivery device (e.g., an autoinjector), a pre-filled medicament container (e.g., syringe) is housed in a carrier which is axially movable to achieve needle penetration in an injection site and, optionally, needle withdrawal. A conventional carrier is typically sized and shaped to retain a medicament container of a predetermined size. In order to increase a dose that can be administered by the delivery device, a larger carrier would be necessary to accommodate a larger medicament container. However, a size and shape of the delivery device may not provide space for the larger medicament container. Thus, an entirely different delivery device may be necessary for different dosage amounts.

Accordingly, there is a need for a carrier and an adapter that allows the carrier to accommodate medicament containers of various sizes.

### SUMMARY OF THE INVENTION

It is an object of the present invention to provide a medicament container carrier and an adapter for the medicament container carrier.

In an exemplary embodiment, a medicament container carrier adapted to hold a first medicament container having a first predetermined size and capable of containing more than a first volume of a medicament according to the present invention comprises a body, and an adapter coupled to the body. The adapter is adapted to hold a second medicament container having a second predetermined size different from the first predetermined size and capable of containing no more than the first volume of the medicament.

In an exemplary embodiment, the first volume is approximately 2 mL.

In an exemplary embodiment, the adapter includes a distal rib adapted to abut a distal end of the second medicament container.

In an exemplary embodiment, the adapter includes at least one opening providing visual access to the second medicament container.

In an exemplary embodiment, the adapter includes at least one resilient arm adapted to engage the second medicament container. The arm includes a first ramp adapted to engage a distal face of a flange to deflect the arm in a first radial direction. The arm includes a first abutment surface adapted to engage the flange to prevent rotation of the second medicament container relative to the adapter and a second abutment surface adapted to engage the flange to prevent axial displacement of the second medicament container relative to the adapter. The arm includes a second ramp adapted to engage an engagement element on the body to deflect the arm in a second radial direction. The arm includes a third abutment surface adapted to abut a proximal stop in the body to prevent axial displacement of the adapter relative to the body, and a fourth abutment surface adapted to engage a restricting surface in the body to prevent rotation of the adapter relative to the body. The proximal stop is formed in an aperture on the body.

In an exemplary embodiment, the first medicament container and the second medicament container are each one of a pre-filled syringe and a medicament cartridge.

In an exemplary embodiment, a medicament delivery device according to the present invention comprises a housing, and a medicament container carrier according to the exemplary embodiments described herein. The medicament container carrier may be slidably disposed in the housing.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, do not limit the present invention, and wherein:

FIG. 1 is a schematic perspective exploded view of an exemplary embodiment of a container carrier and a first medicament container according to the present invention,

FIG. 2 is a schematic perspective view of an exemplary embodiment of a container carrier coupled to a first medicament container according to the present invention,

FIG. 3 is a schematic perspective exploded view of an exemplary embodiment of a container carrier, an adapter and a second medicament container according to the present invention,

FIG. 4 is a schematic perspective view of an exemplary embodiment of an adapter according to the present invention,

FIG. 5 is a schematic perspective view of an exemplary embodiment of a second medicament container coupled to an adapter according to the present invention,

FIG. 6 is a schematic perspective view of an exemplary embodiment of a container carrier coupled to an adapter and a second medicament container according to the present invention, and

FIG. 7 is a schematic perspective detail view of an exemplary embodiment of a container carrier coupled to an adapter and a second medicament container according to the present invention.

Corresponding parts are marked with the same reference symbols in all figures.

### DETAILED DESCRIPTION

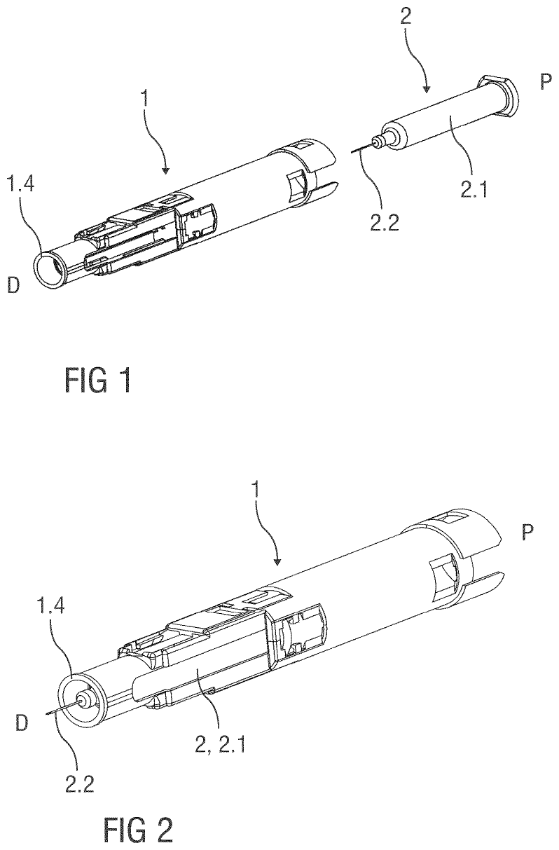

FIG. 1 is a schematic perspective exploded view of an exemplary embodiment of a container carrier 1 having a body adapted to hold a first medicament container 2 according to the present invention. The first medicament container 2, which may be a syringe, an ampoule, a cartridge, etc., has a first predetermined size (e.g., length, diameter, etc.) and contains up to a first maximum amount of a medicament. For example, the first medicament container 2 may be a pre-filled syringe containing a 2 mL dose of a medicament.

In an exemplary embodiment, the first medicament container 2 comprises a medicament container body 2.1, a needle 2.2 mounted to a distal end of the medicament container body 2.1 and in fluid communication with a cavity defined within the medicament container body 2.1 for containing a medicament, and a stopper (not illustrated) disposed within the cavity near a proximal end of the medicament container body 2.1, wherein the stopper proximally seals the cavity and can be axially displaced within the cavity for expelling the medicament through the needle 2.2. The needle 2.2 may fixed to the container body 2.1 or may be removably coupled thereto. In the latter exemplary embodiment, the needle 2.2 may be a double-tipped needle and the distal end of the body 2.1 may include a septum which receives a proximal tip of the needle 2.2. In the figures, “D” refers to the distal direction, and “P” refers to the proximal direction.

FIG. 2 is a schematic perspective view of an exemplary embodiment of the container carrier 1 with the first medicament container 2 coupled thereto. The first medicament container 2 may be inserted into a proximal opening of the container carrier 1 and moved distally until it abuts a distal stop. A retention mechanism (e.g., snaps, clips, locks, etc.) in the container carrier 1 may engage a proximal end (e.g., flange(s)) of the first medicament container 2 to prevent the first medicament container 2 from translating axially relative to the container carrier 1 and/or rotating relative to the container carrier 1.

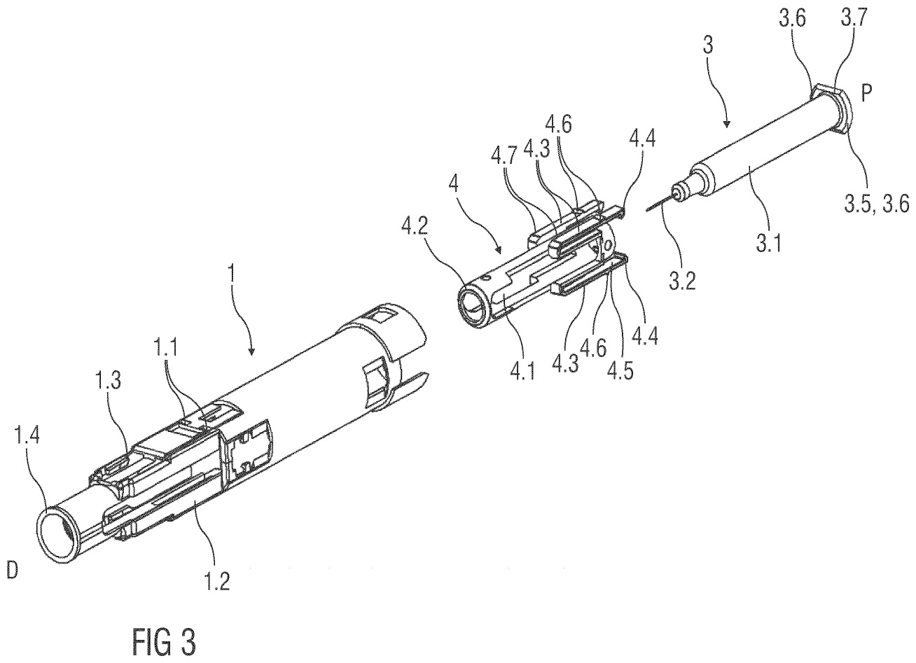

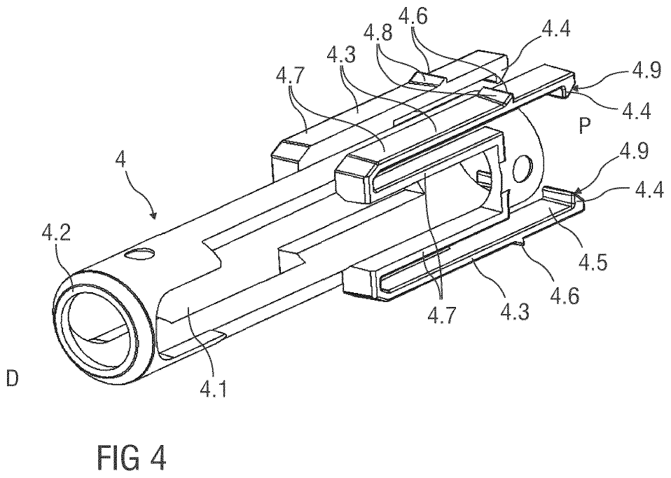

FIG. 3 is a schematic perspective exploded view of an exemplary embodiment of the container carrier 1, an adapter 4 and a second medicament container 3. FIG. 4 is a schematic perspective view of an exemplary embodiment of the adapter 4.

In an exemplary embodiment, the adapter 4 is designed to engage a second medicament container 3 having a second predetermined size smaller than the first predetermined size of the first medicament container 2. The adapter 4 may further be designed to engage the container carrier 1. The second medicament container 3 comprises a medicament container body 3.1, a needle 3.2 mounted to a distal end of the medicament container body 3.1 and in fluid communication with a cavity defined within the medicament container body 3.1 for containing a medicament, and a stopper (not illustrated) disposed within the cavity near a proximal end of the medicament container body 3.1, wherein the stopper proximally seals the cavity and can be axially displaced within the cavity for expelling the medicament through the needle 3.2. The needle 3.2 may fixed to the container body 3.1 or may be removably coupled thereto. In the latter exemplary embodiment, the needle 3.2 may be a double-tipped needle and the distal end of the body 3.1 may include a septum which receives a proximal tip of the needle 3.2. A flange 3.5 may be arranged at a proximal end of medicament container body 3.1. In an exemplary embodiment, the flange 3.5 comprises two arc sections 3.6 connected by two straight sections 3.7.

In an exemplary embodiment, the adapter 4 may be used to allow a medicament delivery device (e.g., a pen injector, an auto-injector, etc.) to accommodate various medicament container sizes. The adapter 4 may allow the usage of a common delivery device for different medicament container sizes. Therefore, a reduction in device costs and consistency in medicament administration may be achieved.

In an exemplary embodiment, the adapter 4 is adapted to retain the second medicament container 3 such that the second medicament container 3 is axially and rotationally fixed within the adapter 4. The adapter 4 is also adapted to be retained within the container carrier 1 such that the adapter 4 is axially and rotationally fixed within the container carrier 1. Preventing rotation of the second medicament container 3 prevents the needle 3.2 from rotating during an injection thus reducing the risk for bending and distorting the needle 3.2 which may otherwise result in pain. Preventing axial movement of the medicament container 3 in a proximal direction P also prevents the second medicament container 3 from being pushed in the proximal direction P relative to the adapter 4 and the container carrier 1 upon contact of the needle 3.2 with an injection site.

In an exemplary embodiment as shown in FIG. 4, the adapter 4 may be sized and/or shaped to telescopically engage the container carrier 1 and the second medicament container 3. For example, the adapter 4 may have an outer geometry which corresponds to an inner shape of the container carrier 1 and an inner geometry which corresponds to the second predetermined size/shape of the second medicament container 3. A distal rib 4.2 formed on a distal end of the adapter 4 may be adapted to abut the distal end of the container body 3.1 to prevent distal movement of the second medicament container 3 beyond the distal rib 4.2.

In an exemplary embodiment, the adapter 4 may include one or more lateral openings 4.1 for allowing inspection of the contents and state of the second medicament container 3. In another exemplary embodiment, the adapter 4 may be made from a transparent/translucent material to provide visual access to the contents of the second medicament container 3.

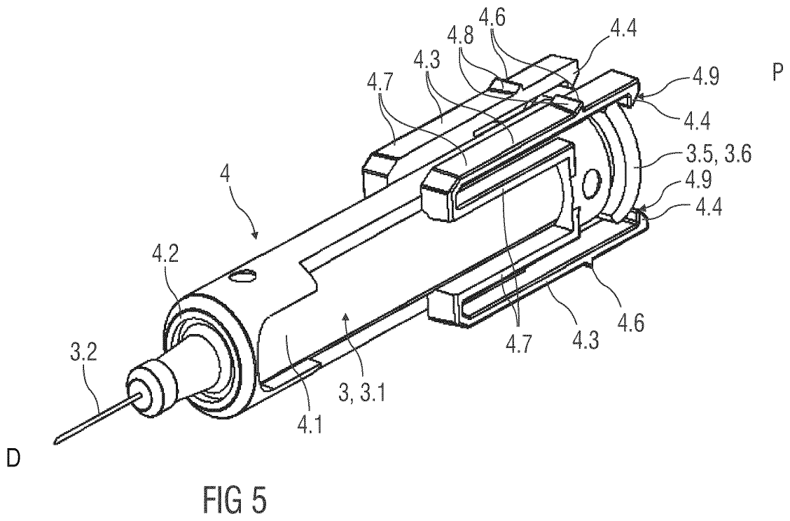

In an exemplary embodiment, the adapter 4 may comprise at least one resilient arm 4.3 extending proximally and adapted to engage a proximal portion (e.g., the flange 3.5) of the container body 3.1. In the exemplary embodiment shown in FIG. 4, the adapter 4 includes four resilient arms 4.3. The arm 4.3 may include features to facilitate insertion and retention of the second medicament container 3. For example, a proximal end of the arm 4.3 may include a first ramp 4.9 which is adapted to engage the flange 3.5 (e.g., the straight section 3.7) when the second medicament container 3 is inserted into the adapter 4. As the flange 3.5 engages the first ramp 4.9, the arm 4.3 is deflected in a first radial direction until the flange 3.5 bypasses the ramped face 4.9. As shown in FIG. 5, when the arm 4.3 returns to a non-deflected position, a first abutment surface 4.5 may engage the flange 3.5 (e.g., straight section 3.7) and thereby prevent the second medicament container 3 from rotating relative to the adapter 4, and a second abutment surface 4.4 may abut a proximal face of the flange 3.5 and thereby prevent the second medicament container 3 from moving axially relative to the adapter 4.

Referring back to the exemplary embodiment shown in FIG. 4, the resilient arm 4.3 may include a second ramp 4.8, a third abutment surface 4.6, and a fourth abutment surface 4.7 which facilitate insertion and retention of the adapter 4 by the container carrier 1. For example, the second ramp 4.8 may be disposed on a radially outer surface of the arm 4.3. When the adapter 4 is inserted into the container carrier 1, the second ramp 4.8 may engage an element or an aperture 1.1 on the container carrier 1, which causes the arm 4.3 to deflect in a second radial direction (opposite the first radial direction) until the second ramp 4.8 bypasses the element. When the arm 4.3 returns to the non-deflected position, the third abutment surface 4.6 may engage a proximal stop in the container carrier 1 and thereby prevent axial displacement of the adapter 4 relative to the container carrier 1. The fourth abutment surface 4.7 may engage a surface or element in the container carrier 1 to prevent rotation of the adapter 4 relative to the container carrier 1.

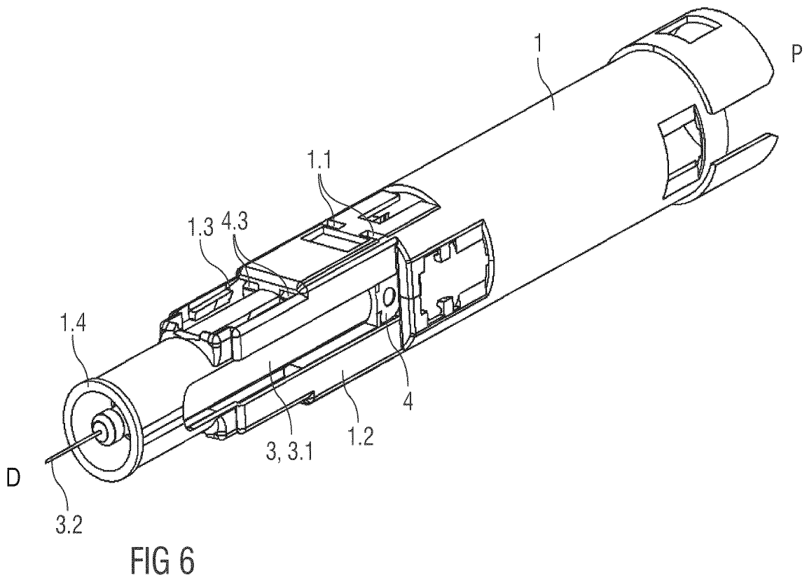

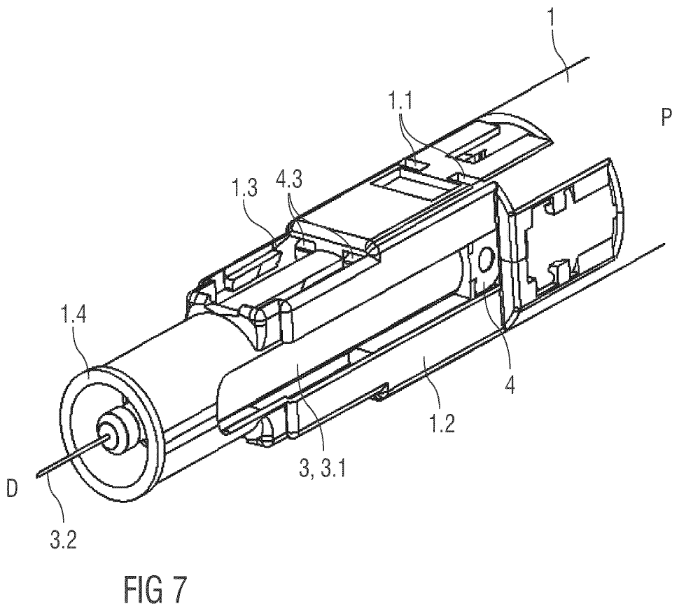

The second medicament container 3 may be assembled with the container carrier 1 as shown in FIGS. 6 and 7 in at least two ways. In a first exemplary process, the adapter 4 may be inserted into the container carrier 1 and translated distally until it abuts a distal stop 1.3 and the third abutment surface 4.6 abuts proximal stop. The second medicament container 3 may then be inserted into the adapter 4 and translated distally until it abuts the distal rib 4.2 and the second abutment surface 4.4 abuts the flange 3.5. In a second exemplary process, the second medicament container 3 may be inserted into the adapter 4, and the adapter 4 may then be inserted into the container carrier 1.

In an exemplary embodiment, the first medicament container 2 may be adapted to contain greater than approximately 2 mL of a medicament, and the second medicament container may be adapted to contain less than approximately 2 mL of a medicament.

Those of skill in the art will understand that modifications (additions and/or removals) of various components of the apparatuses, methods and/or systems and embodiments described herein may be made without departing from the full scope and spirit of the present invention, which encompass such modifications and any and all equivalents thereof.

1. (canceled)

2. An auto-injector comprising:
a housing; and
a translucent syringe support configured to be disposed in the housing, the translucent syringe support comprising:
one or more first surfaces configured to engage diametrically opposite portions of a proximal side of a proximal flange of a syringe to limit proximal movement of the syringe relative to the one or more first surfaces when the proximal flange of the syringe is disposed in the translucent syringe support; and
a second surface that is distal to a distal side of the proximal flange of the syringe and configured to limit distal movement of the syringe relative to the second surface when the proximal flange of the syringe is disposed in the translucent syringe support,
wherein the auto-injector is configured to dispense a volume of a medicament from the syringe.

3. The auto-injector of claim 2, comprising the syringe.

4. The auto-injector of claim 3, wherein the volume of the medicament is greater than 0 milliliters and less than 2 milliliters.

5. The auto-injector of claim 2, wherein the translucent syringe support comprises a pair of third surfaces configured to limit a rotation of the syringe relative to the housing, the pair of third surfaces facing one another and being perpendicular to the one or more first surfaces.

6. The auto-injector of claim 5, wherein the pair of third surfaces of the translucent syringe support are configured to directly contact the syringe.

7. The auto-injector of claim 2, wherein the second surface of the translucent syringe support is configured to directly contact the syringe.

8. The auto-injector of claim 2, wherein the distal side of the proximal flange of the syringe is proximally spaced from the translucent syringe support when the proximal flange of the syringe is disposed in the translucent syringe support between the one or more first surfaces and the second surface.

9. The auto-injector of claim 2, wherein the translucent syringe support comprises an axially-extending portion configured to be located radially outward of the proximal flange of the syringe when the syringe is disposed in the translucent syringe support, the axially-extending portion extending from the distal side of the proximal flange of the syringe to the proximal side of the proximal flange of the syringe.

10. The auto-injector of claim 8, wherein the translucent syringe support comprises a proximal portion extending radially inward from a proximal end of the axially-extending portion on the proximal side of the proximal flange when the syringe is disposed in the translucent syringe support, the proximal portion defining the one or more first surfaces.

11. The auto-injector of claim 9, wherein the one or more first surfaces extend in a plane that is perpendicular to a longitudinal axis of the syringe along a substantial entirety of the one or more first surfaces when the syringe is disposed in the translucent syringe support.

12. A translucent syringe support for a syringe, the translucent syringe support comprising:
one or more first surfaces configured to engage diametrically opposite portions of a proximal side of a proximal flange of the syringe to limit proximal movement of the syringe relative to the translucent syringe support when the syringe is disposed in the translucent syringe support; and
a second surface that is distal to a distal side of the proximal flange of the syringe to limit distal movement of the syringe relative to the translucent syringe support when the syringe is disposed in the translucent syringe support.

13. The translucent syringe support of claim 12, comprising an axially-extending portion configured to be radially outward of the proximal flange of the syringe and extending from the distal side of the proximal flange of the syringe to the proximal side of the proximal flange of the syringe when the syringe is disposed in the translucent syringe support.

14. The translucent syringe support of claim 13, comprising a proximal portion extending radially inward from a proximal end of the axially-extending portion on the proximal side of the proximal flange, a distal side of the proximal portion defining the one or more first surfaces.

15. The translucent syringe support of claim 14, wherein a substantial entirety of the one or more first surfaces extend in a plane that is perpendicular to a longitudinal axis of the syringe.

16. The translucent syringe support of claim 12, wherein the translucent syringe support comprises a pair of third surfaces.

17. The translucent syringe support of claim 16, wherein the pair of third surfaces face one another and are perpendicular to the one or more first surfaces.

18. The translucent syringe support of claim 17, wherein the pair of third surfaces of the translucent syringe support are configured to directly contact the syringe.

19. The translucent syringe support of claim 12, wherein the distal side of the proximal flange of the syringe is proximally spaced from the translucent syringe support when the syringe is disposed in the translucent syringe support with the proximal side of the proximal flange of the syringe engaged to the one or more first surfaces.

20. The translucent syringe support of claim 19, wherein the second surface of the translucent syringe support is configured to directly contact the syringe.

21. The translucent syringe support of claim 12, wherein the syringe is disposed in the translucent syringe support with the proximal flange axially between one or more first surfaces and the second surface.

22. The translucent syringe support of claim 21, wherein the syringe contains a volume of a medicament greater than 0 milliliters and less than 2 milliliters.
